(19) United States
(12) Patent Application Publication
(10) Pub. No.: US 2016/0058401 A1
(43) Pub. Date: Mar. 3, 2016

(54) MR-PET Hybrid Scout Scan/Topogram Workflow

(71) Applicant: Siemens Medical Solutions USA, Inc., Malvern, PA (US)

(72) Inventors: James Frank Caruba, Bartlett, IL (US); Ralf Ladebeck, Erlangen (DE)

(21) Appl. No.: 14/472,417

(22) Filed: Aug. 29, 2014

(51) Int. Cl. A61B 6/00; A61B 6/03; A61B 6/04; A61B 5/055

(52) U.S. Cl. CPC A61B 6/4417; A61B 5/055; A61B 6/037; A61B 6/5247; A61B 6/0407

(57) ABSTRACT

A magnetic resonance and positron emission tomography (MR-PET) hybrid scanner workflow for providing a scout scan (topogram) of a patient includes (a) positioning the patient in the MR-PET hybrid scanner; (b) referencing the patient to the scanner's field of view; (c) specifying the patient parameters for MR and PET hybrid topogram scan; (d) performing hybrid MR-PET topogram scan and acquiring MR topogram scan data and PET topogram scan data simultaneously; and (e) reconstructing the MR and PET topogram scan images in real-time; and (f) presenting the topogram san images as a fused topogram data set.

### FIELD

The present disclosure generally relates to a method for acquiring scout scan data for producing a topogram in a hybrid combination magnetic resonance and positron emission tomography (MR-PET) scanning systems, and more particularly to a hybrid MR-PET scout scan/topogram workflow.

### BACKGROUND

MR-PET hybrid scanning is an attractive hybrid imaging modality which has gained significant attention in recent years. MR, PET, and CT imaging modalities have the ability to perform planar imaging using computed tomography-based data sets taken under low dose/fast acquisition conditions for patient scouting purposes which are commonly referred to as a topogram or scout scan. The topogram or scout scan provides a general scan of a desired examination region of a body which is to be examined. The topogram is a parallel or central projection of the body which reproduces the anatomic structure of the body.

In current MR-PET hybrid imaging systems, one of two methods are used for the scout scan for planning the patient examination. In some current MR-PET hybrid systems, MR-only image is used for the scout scan planning. In other MR-PET hybrid systems, the scout scans are performed independently on separate scanners or serially within a single hybrid scanner with post-scan data fusion. In such scout scan workflow, patient's body can move between the scout scan and the subsequent diagnostic scan. Therefore, in the current MR-PET hybrid systems, patient motion between scans on separate scanners or between the serial scans on a single hybrid scanner requires extra workflow steps to support registration of images or image fusion. Additionally, in the current hybrid systems, one has no knowledge of PET lesion localization during the planning of the patient examination.

Some general information about MR-PET hybrid imaging systems may be found in U.S. Pat. No. 7,218,112 to Ladebeck et al. and U.S. Pat. No. 8,467,847 to James Frank Caruba et al., both of which are incorporated herein by reference in their entirety.

### SUMMARY

The inventors hereby disclose a radiology workflow that is optimized and streamlined for maximum information content and hybrid MR-PET scanner utilization. The optimized workflow provides a method for providing a topogram of a patient in a MR-PET hybrid scanner, where the method comprises: positioning the patient in the MR-PET hybrid scanner; referencing the patient to the scanner's field of view; specifying the patient height and other MR and PET hybrid topogram scan parameters; launching hybrid MR-PET scout/topogram scan using a multi-station stop and shoot approach or continuous table moving acquisition; acquiring MR and PET scan data simultaneously; and reconstructing the MR and PET images in real-time and presenting them as a fused topogram data set.

The disclosed invention provides a hybrid MR-PET scout/topogram workflow with both PET and MR acquisitions performed simultaneously with the image data displayed together and co-registered in one topogram. The invention provides an enhanced scout/topogram with more inherent diagnostic information by simultaneous acquisition of MR-PET scan data and reconstructed topogram that presents both MR and PET data co-registered.

### BRIEF DESCRIPTION OF THE DRAWINGS

The following will be apparent from elements of the figures, which are provided for illustrative purposes and are not necessarily to scale.

FIG. 1 is a flowchart illustrating the disclosed MR-PET hybrid scanner's scout/topogram workflow.

FIG. 2 is a perspective view schematic illustration of a MR-PET hybrid scanner.

FIG. 3 is a plan view schematic illustration of the MR-PET hybrid scanner of FIG. 2.

### DETAILED DESCRIPTION

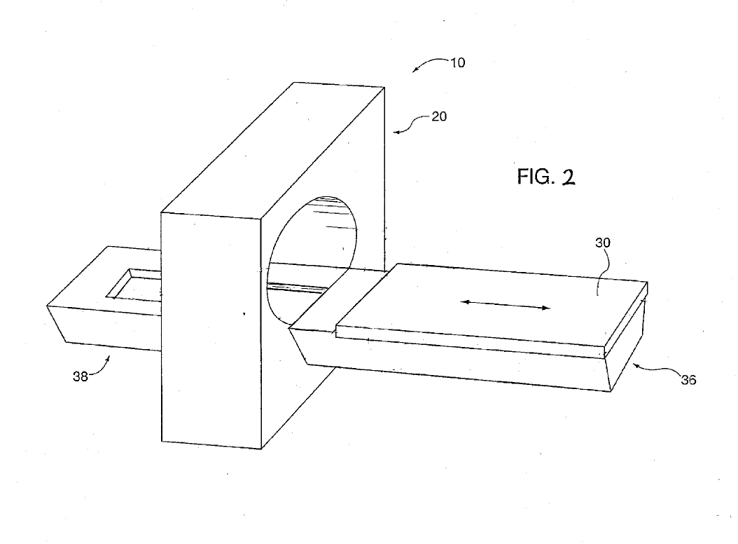

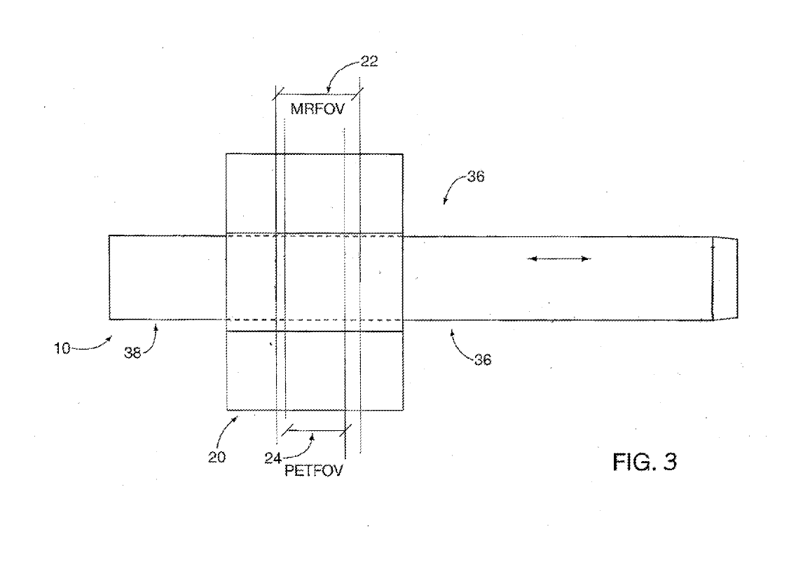

FIGS. 2 and 3 show generally a MR-PET hybrid scanner 10 having a scanner gantry 20 that incorporates scanner components necessary to acquire patient images in both the MR and PET modalities. In this example of MR-PET hybrid scanner, the MR and PET scan fields of view (FOV), respectively 22, 24, are overlapping.

The MR-PET hybrid scanner 10 includes a patient table 30. A front patient table support 36 supports the patient table 30 on one side of the scanner and a rear patient table support 38 supports the patient table 30 on the other side of the scanner. The patient table 30 translates from a patient loading area on the front table support 36 side of the scanner 10, where the table is entirely outside of the MR and PET FOVs 22, 24. A patient is placed on the patient table 30 and thereafter the patient table is linearly translated so that the front end (the head side) of the patient table 30 penetrates the FOVs 22, 24 so that the portion of the patient's anatomy to be scanned is positioned in the relevant FOV for scanning.

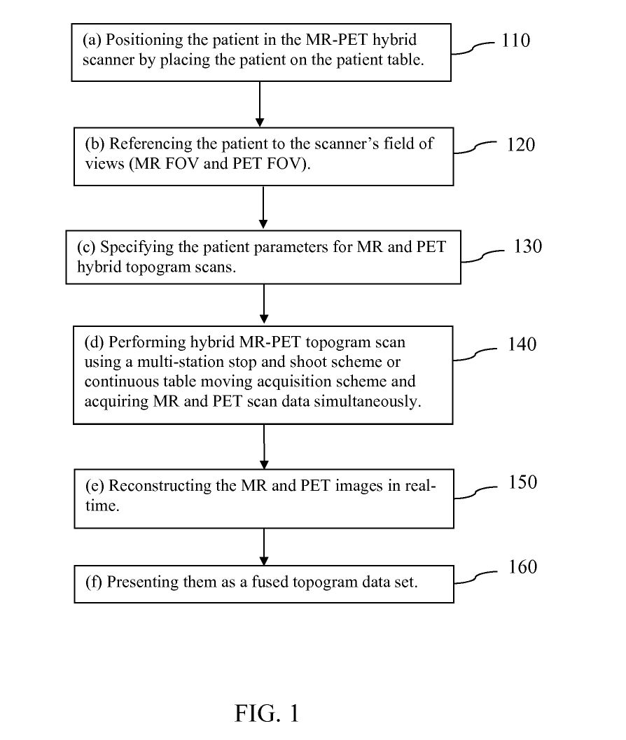

A radiology workflow according to an embodiment of the present disclosure is shown in the flowchart 100 of FIG. 1. The workflow will be described with reference to the MR-PET hybrid scanner 10 of FIGS. 2 and 3. The workflow for the MR-PET hybrid scanner 10 provides a method for providing a topogram of a patient in the MR-PET hybrid scanner. The method comprises first positioning the patient in the MR-PET hybrid scanner 10 by placing the patient on the patient table 30. (See block 110). Next, the patient's body is referenced to the scanner's FOVs 22, 24. (See block 120). Referencing the patient's body is accomplished by positioning the patient table to a reference point identified by the scanner laser cross hairs that are projected onto the patient's body.

Next, patient parameters for MR and PET hybrid topogram scans are defined for the MR-PET hybrid scanner. (See block 130). Examples of patient parameters are patient height, the scan orientation (transversal and/or coronal and/or sagittal) for MR scan, and the speed of the patient table in cases where the patient table moves continuously. The technician operating the MR-PET hybrid scanner 10 enters these scan parameters to the hybrid scanner's controller.

Next, a hybrid MR-PET topogram scan is performed by either a multi-station stop and shoot scheme or a continuous patient table moving acquisition scheme. (See block 140). Multi-station stop and shoot is an acquisition scheme where the scan data is collected at intervals while the patient table is moved in discrete steps, the scan being performed when the patient table is stationary between each steps. Each discrete step taken can be of any desired distance so that the region scanned on the patient can be adjacent, overlapping, or separated. Continuous table moving acquisition is an acquisition scheme where the scan data acquisition starts in a first patient table position A, dwells for a start time at the position A, moves at a constant velocity to position B, dwells for a stop time at position B and then concludes the scan acquisition at the end of the dwell period. During the hybrid MR-PET topogram scanning, MR topogram scan data and PET topogram scan data are simultaneously acquired utilizing the MR-PET hybrid scanners' MR and PET scanning systems.

Next, the MR-PET hybrid scanner's controller reconstructs the MR and PET images in real-time and presents them as one fused topogram data set. (See block 150). The reconstruction of scan images for each modalities, MR and PET, is conducted by the reconstruction methods and algorithms already known to those skilled in the art.

The disclosed invention provides a hybrid MR-PET scout/topogram workflow with both PET and MR acquisitions performed simultaneously with the image data displayed together and co-registered in one topogram. The invention provides an enhanced scout/topogram with more inherent diagnostic information by simultaneous acquisition of MR-PET scan data and reconstructed topogram that presents both MR and PET data co-registered. For example, edge of PET lesion localization during the planning of the patient examination

The embodiments and examples set forth herein are presented to best explain the present disclosure and its practical application and to thereby enable those skilled in the art to make and utilize the present disclosure. However, those skilled in the art will recognize that the foregoing description and examples have been presented for the purpose of illustration and example only. Thus, while preferred embodiments of the present disclosure have been described, it is to be understood that the embodiments described are illustrative only and that the scope of the invention is to be defined solely by the appended claims when accorded a full range of equivalence, many variations and modifications naturally occurring to those of skill in the art from a perusal hereof.

What is claimed is:

1. A method for providing a topogram of a patient in a magnetic resonance and positron emission tomography (MR-PET) hybrid scanner, the method comprising:
(a) positioning the patient in the MR-PET hybrid scanner;
(b) referencing the patient to the scanner's field of view;
(c) specifying the patient parameters for MR and PET hybrid topogram scan;
(d) performing hybrid MR-PET topogram scan and acquiring MR topogram scan data and PET topogram scan data simultaneously;
(e) reconstructing the MR and PET topogram scan images in real-time; and
(f) presenting the MR and PET topogram scan images as a fused topogram data set.

2. The method of claim 1, wherein the step of performing hybrid MR-PET topogram scan comprises using a multi-station stop and shoot scheme.

3. The method of claim 1, wherein the step of launching hybrid MR-PET topogram scan comprises using a continuous patient table moving acquisition scheme.
